SEQUENCE LISTING (3 sequences)

SEQ ID NO: 1 - DNA, 1884 bp, Arthrobacter sp.
  CDS (35)..(1384)
  mat_peptide (155)..(1384)
ctgcagcagtcttatcccatacaaaggagaccccgtggtaactggcaagaatctagacaaagcgaatccaagccgccgtcggctgatcggcgccggagccgccggaaccctggcggctgccttgaccctcgggacgatgcagaacgccaatgcggccgacggccagcaaggtacccccctcaattcgcccaacacgtacgacgtaaccacatggaggatcaaggcacacccggacgtcaccgcgcagtccgacattggggcggtcatcaacgacatcatcgccgacatcaagcaacggcagacgtcaccggacgcgcgtcccggagccgcgatcattatcccaccgggcgactacgacctgcacacccaggtcgtcgtcgacataagttacctgacaatcgcgggcttcgggcatggcttcttctcccgaagcatcctcgacaactcgaacccgaccggatggcagaacctccaacccggagcaagccacatccgcgtcctgacctctccgagcgcgccccaggcattcctcgtccgccggacaggggatccccgtctttcaggaatcgtgttccgggacttctgcctcgacggagtcggcttcacccccgacaagaacagctaccacaacggcaagaccggaatcgaagtcgcctccgacaacgactccttccacatcaccggcatgggattcgtctacctcgaacatgccctgatcgtgcgcggcgccgacgcgctccgcgtcaacgacaacatgatcgccgaatgcggcaactgcgtcgagctcaccggggccgggcaggccacaattgtcagcggcaatcacatgggcgccggccctgacggggtaaccctcctggccgagaaccacgagggcctcctcgtcaccggcaacaacctcttcccacgcggccgcagcctcatcgaactcaccggctgcaaccggtcctcagtctcctcgaacaggctccagggcttttacccgggcatgctccgcctgctgaacggctgcaaggagaacctcatcacggccaaccacatccgccggaccaacgaggggtacccgccgttcatcggccgcggcaacggcctcgacgacctctacggcgtcgtccacatcgcgggagacaacaacctcatctcggacaacctcttcgcctacaacgtcccgcccggcaacatcgcccccgccggcgcccagccgacccagatcctgatcgccggcggagacgccaacgtggtggcgctcaaccacgtggtcagcgacgtcgcttcccagcacgtcgttctggacgcatccaccactcactcgaaagtgctcgacagcggtaccgcctcccagatcacctcgtacagcacggacaccgctatccggccgaccccctgacaggcggagagcagcttctcggaaaccaccggacgcgccaagggcatttcttatgttggggcccggaccaatcggtgatatcgcggggagcctcagcggtccttgagaggctccccgatcaattcgggctgccggttgctccagtcgtggaagtagggagcggcgccgtggtggtgcttgttgttgtactcctgggcaagacccagtgcaccttcgagcccggggaagacccggtctttggtgtgatcagcgcatctgacgaggaaaccgagccccctaaagccgtagcactgggttacataagcgggtcgagtcgaaatgtcccccttggtgtcgttccgccctccgacggggcccgcttagatggttctatctccggaatcctgatctacctcagtcactggtgatttgatccatgtgacgaccacactcaccccgccgtcctcgtcccgttcggtctcgatttcaatctcggaagccgacgccccaataagctt

SEQ ID NO: 2 - DNA, 1737 bp, Arthrobacter sp.
  CDS (1)..(255)
  mat_peptide (25)..(255)
atgaccatgattacgccaagcttggccgacggccagcaaggtacccccctcaattcgcccaacacgtacgacgtaaccacatggaggatcaaggcacacccggacgtcaccgcgcagtccgacattggggcggtcatcaacgacatcatcgccgacatcaagcaacggcagacgtcaccggacgcgcgtcccggagccgcgatcattatcccaccgggcgactacgacctgcacacccaggtcgtcgtcgacataagttacctgacaatcgcgggcttcgggcatggcttcttctcccgaagcatcctcgacaactcgaacccgaccggatggcagaacctccaacccggagcaagccacatccgcgtcctgacctctccgagcgcgccccaggcattcctcgtccgccggacaggggatccccgtctttcaggaatcgtgttccgggacttctgcctcgacggagtcggcttcacccccgacaagaacagctaccacaacggcaagaccggaatcgaagtcgcctccgacaacgactccttccacatcaccggcatgggattcgtctacctcgaacatgccctgatcgtgcgcggcgccgacgcgctccgcgtcaacgacaacatgatcgccgaatgcggcaactgcgtcgagctcaccggggccgggcaggccacaattgtcagcggcaatcacatgggcgccggccctgacggggtaaccctcctggccgagaaccacgagggcctcctcgtcaccggcaacaacctcttcccacgcggccgcagcctcatcgaactcaccggctgcaaccggtcctcagtctcctcgaacaggctccagggcttttacccgggcatgctccgcctgctgaacggctgcaaggagaacctcatcacggccaaccacatccgccggaccaacgaggggtacccgccgttcatcggccgcggcaacggcctcgacgacctctacggcgtcgtccacatcgcgggagacaacaacctcatctcggacaacctcttcgcctacaacgtcccgcccggcaacatcgcccccgccggcgcccagccgacccagatcctgatcgccggcggagacgccaacgtggtggcgctcaaccacgtggtcagcgacgtcgcttcccagcacgtcgttctggacgcatccaccactcactcgaaagtgctcgacagcggtaccgcctcccagatcacctcgtacagcacggacaccgctatccggccgaccccctgacaggcggagagcagcttctcggaaaccaccggacgcgccaagggcatttcttatgttggggcccggaccaatcggtgatatcgcggggagcctcagcggtccttgagaggctccccgatcaattcgggctgccggttgctccagtcgtggaagtagggagcggcgccgtggtggtgcttgttgttgtactcctgggcaagacccagtgcaccttcgagcccggggaagacccggtctttggtgtgatcagcgcatctgacgaggaaaccgagccccctaaagccgtagcactgggttacataagcgggtcgagtcgaaatgtcccccttggtgtcgttccgccctccgacggggcccgcttagatggttctatctccggaatcctgatctacctcagtcactggtgatttgatccatgtgacgaccacactcaccccgccgtcctcgtcccgttcggtctcgatttcaatctcggaattc

SEQ ID NO: 3 - DNA, 1737 bp, Arthrobacter sp.
atgaccatgattacgccaagcttggccgacggccagcaaggtacccccctcaattcgcccaacacgtacgacgtaaccacatggaggatcaaggcacacccggacgtcaccgcgcagtccgacattggggcggtcatcaacgacatcatcgccgacatcaagcaacggcagacgtcaccggacgcgcgtcccggagccgcgatcattatcccaccgggcgactacgacctgcacacccaggtcgtcgtcgacataagttacctgacaatcgcgggcttcgggcatggcttcttctcccgaagcatcctcgacaactcgaacccgaccggatggcagaacctccaacccggagcaagccacatccgcgtcctgacctctccgagcgcgccccaggcattcctcgtccgccggacaggggatccccgtctttcaggaatcgtgttccgggacttctgcctcgacggagtcggcttcacccccgacaagaacagctaccacaacggcaagaccggaatcgaagtcgcctccgacaacgactccttccacatcaccggcatgggattcgtctacctcgaacatgccctgatcgtgcgcggcgccgacgcgctccgcgtcaacgacaacatgatcgccgaatgcggcaactgcgtcgagctcaccagggccgggcaggccacaattgtcagcggcaatcacatgggcgccggccctgacggggtaaccctcctggccgagaaccacgagggcctcctcgtcaccggcaacaacctcttcccacgcggccgcagcctcatcgaactcaccggctgcaaccggtcctcagtctcctcgaacaggctccagggcttttacccgggcatgctccgcctgctgaacggctgcaaggagaacctcatcacggccaaccacatccgccggaccaacgaggggtacccgccgttcatcggccgcggcaacggcctcgacgacctctacggcgtcgtccacatcgcgggagacaacaacctcatctcggacaacctcttcgcctacaacgtcccgcccggcaacatcgcccccgccggcgcccagccgacccagatcctgatcgccggcggagacgccaacgtggtggcgctcaaccacgtggtcagcgacgtcgcttcccagcacgtcgttctggacgcatccaccactcactcgaaagtgctcgacagcggtaccgcctcccagatcacctcgtacagcacggacaccgctatccggccgaccccctgacaggcggagagcagcttctcggaaaccaccggacgcgccaagggcatttcttatgttggggcccggaccaatcggtgatatcgcggggagcctcagcggtccttgagaggctccccgatcaattcgggctgccggttgctccagtcgtggaagtagggagcggcgccgtggtggtgcttgttgttgtactcctgggcaagacccagtgcaccttcgagcccggggaagacccggtctttggtgtgatcagcgcatctgacgaggaaaccgagccccctaaagccgtagcactgggttacataagcgggtcgagtcgaaatgtcccccttggtgtcgttccgccctccgacggggcccgcttagatggttctatctccggaatcctgatctacctcagtcactggtgatttgatccatgtgacgaccacactcaccccgccgtcctcgtcccgttcggtctcgatttcaatctcggaattc

(19) United States
(12) Patent Application Publication
(10) Pub. No.: US 2004/0091998 A1
(43) Pub. Date: May 13, 2004

(54) Method for microbial production of difructose anhydride III , micro-organism used therefor and enzyme with inulase II activity and dna sequences coding therefor

(76) Inventors: Martin Walter, Bortfeld (DE); Milada Schubert, Vechelde (DE); Klaus-Dieter Vorlop, Braunschweig (DE); Ulrich Jahnz, Braunschweig (DE)

Correspondence Address: SALTER & MICHAELSON, THE HERITAGE BUILDING, 321 SOUTH MAIN STREET, PROVIDENCE, RI, 029037128

(21) Appl. No.: 10/276,751

(22) PCT Filed: May 18, 2001
(86) PCT No.: PCT/EP01/05737

(30) Foreign Application Priority Data
May 19, 2000 (DE) 10024569.2

(51) Int. Cl. C12N 1/20

(52) U.S. Cl. 435/252.3

(57) ABSTRACT

The invention relates to a method for the production of difructose anhydride III by enzymatic decomposition of inulase using an enzyme with inulase II activity. Said enzyme can be obtained from a microorganism of the Arthrobacter sp. Bu0141 species. The invention also relates to DNA sequences derived therefrom comprising a region coding for said enzyme in addition to plasmides and micro-organisms containing said DNA sequences.

The present invention relates to a process for the microbial production of difructose anhydride III, a microorganism which is suitable for this process and has the ability to express an enzyme with inulase II activity, an enzyme with inulase II activity and DNA sequences with a region coding for this enzyme.

Difructose anhydride III is a disaccharide which contains two fructose units linked to one another via 1-2′ and 2-3′ bonds.

Difructose anhydride III (DFA III) can be obtained by microbial decomposition of inulin by the enzyme inulase II, a transferase.

It is known that the enzyme inulase II can be produced by some microorganisms. These include various species of the genus Arthrobacter, such as, for example, Arthrobacter ureafaciens 7116, Arthrobacter globiformis C 11-1, Arthrobacter aurescens IFO 12136 and Arthrobacter ilicis MCI-2297, and of the genus Pseudomonas, such as Pseudomonas fluorescens no. 949.

A process for the microbial decomposition of inulin to DFA III by means of Arthrobacter ilicis is described in EP 0 332 108 B1, the enzyme with inulase II activity obtained from this microorganism showing a maximum activity at 60° C. and being stable for a short time up to a temperature of 70° C. However, there is no information on the period of time and the residual activity.

There was, however, a demand for further improved processes, in particular for processes which can be carried out with easily obtainable and accessible (recombinant) microorganisms, and for enzymes which still have high residual activities, preferably of up to 100%, over a long period of time, for example several hours, even at elevated temperature.

The object of the present invention was therefore to provide a process for the microbial production of difructose anhydride III which can be carried out with easily obtainable accessible microorganisms which can obtain DFA III from inulin with a high efficiency.

It was also an object of the invention to provide an enzyme with inulase II activity which has a high heat stability over a long period of time.

To achieve the object, the present invention provides, in particular, DNA sequences which code for an enzyme with inulase II activity, and microorganisms which contain and can express this gene and which can advantageously be used for a process for the microbial production of DFA III.

The present invention therefore relates to DNA sequences which code for an enzyme with inulase II activity, characterized by that after introduction of these DNA sequences into a microorganism, there occurs expression of the enzyme with inulase II activity which effects the decomposition of inulin to DFA III.

The invention relates in particular to DNA sequences which code for an enzyme with inulase II activity, comprising

a nucleotide sequence according to sequence no. 1, sequence no. 2 or sequence no. 3 as shown in the Figures;

a nucleotide sequence which comprises the region according to one of sequences no. 1 to 3 which codes for an enzyme with inulase II activity, and

a nucleotide sequence which codes for an enzyme which comprises the amino acid sequences shown for sequences no. 1 to 3.

A reproduction of the sequences is to be found in the sequence listing section of the description:

DNA sequence no. 1 with the amino acid sequence derived therefrom.

DNA sequence no. 2 with the amino acid sequence derived therefrom, and

DNA sequence no. 3.

The invention furthermore relates to a microorganism of the genus Arthrobacter which contains one of the abovementioned DNA sequences, and to plasmids and recombinant microorganisms which contain one of the abovementioned DNA sequences.

The invention furthermore relates to a process for the microbial or enzymatic production of difructose anhydride III which is carried out using one of the abovementioned DNA sequences or a plasmid or a microorganism which contains one of the abovementioned DNA sequences.

The Figures show

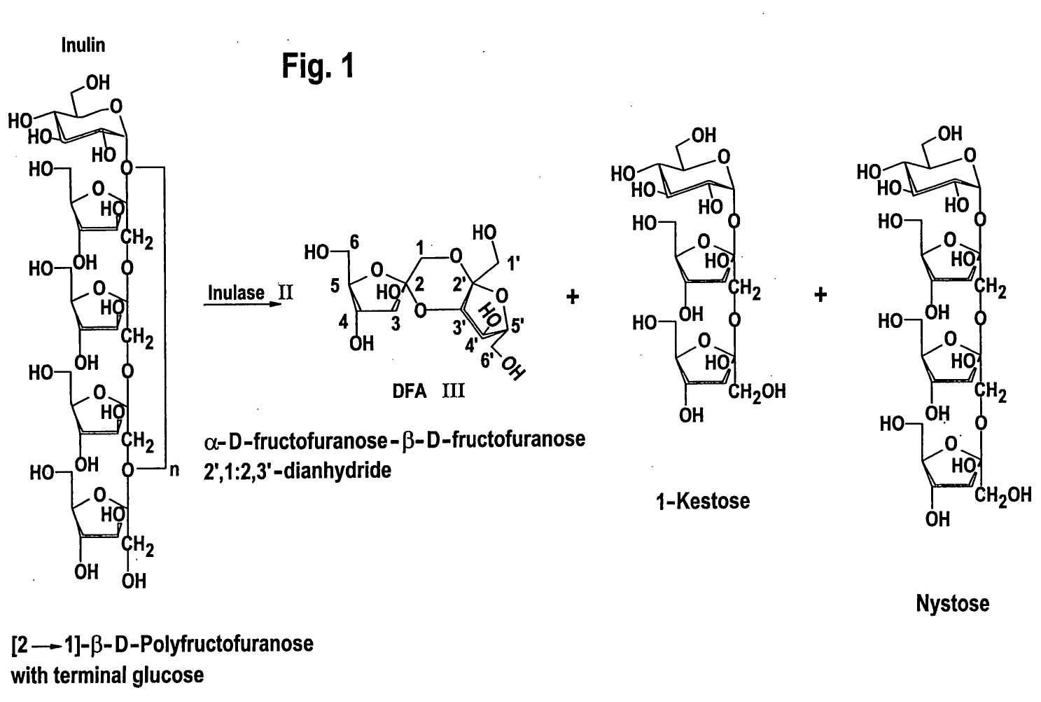

FIG. 1 the enzymatic synthesis of DFA II and fructo-oligosaccharides from inulin;

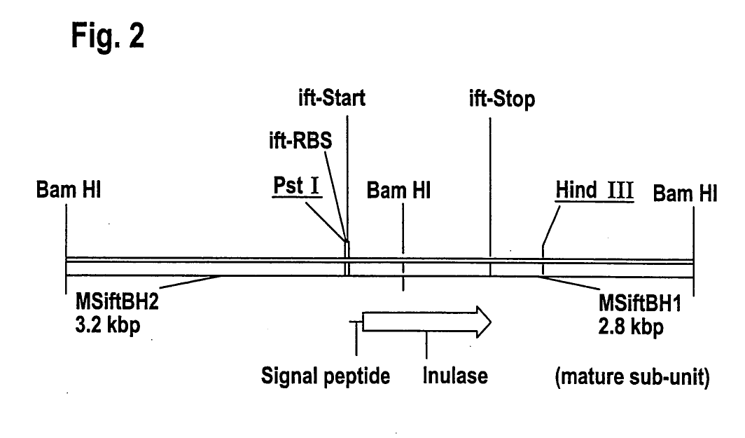

FIG. 2 the gene map of the Bam H1 fragments MSiftBH2 and MSiftBH1 from Arthrobacter Bu0141;

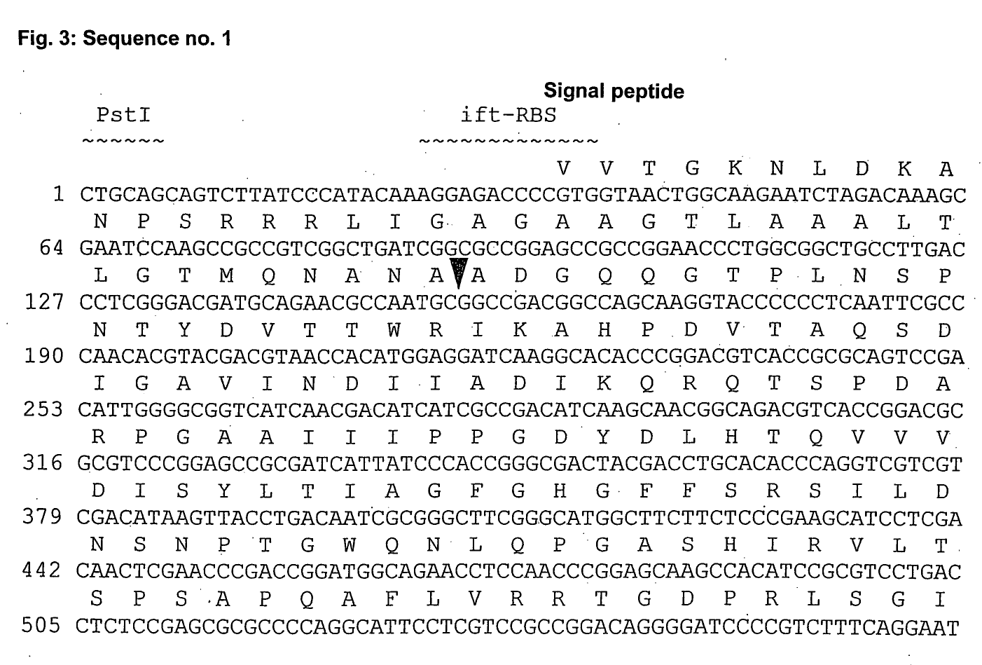

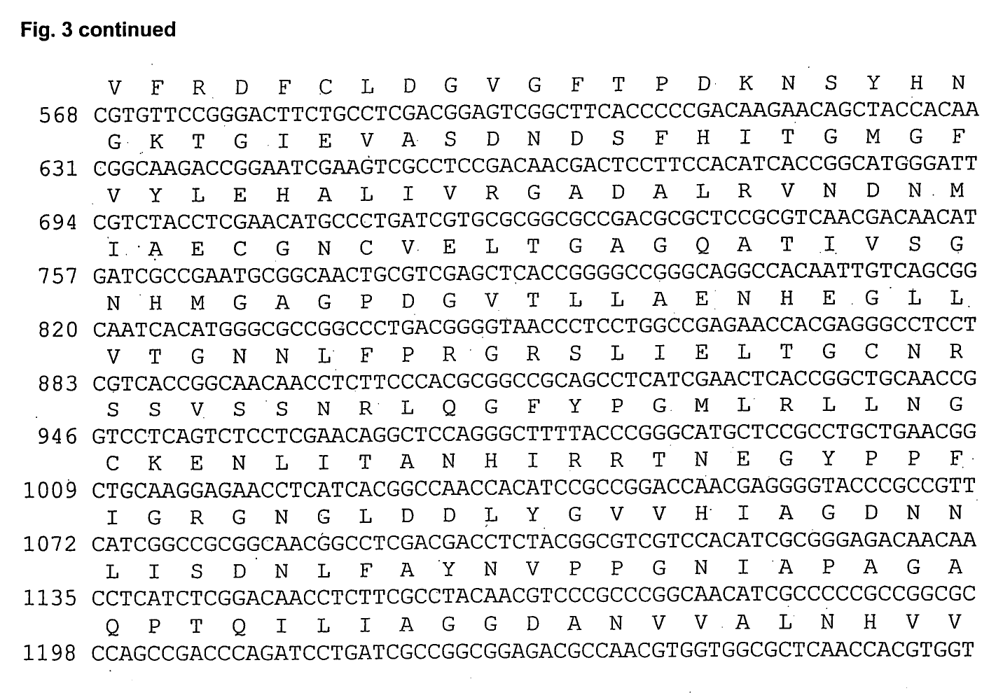

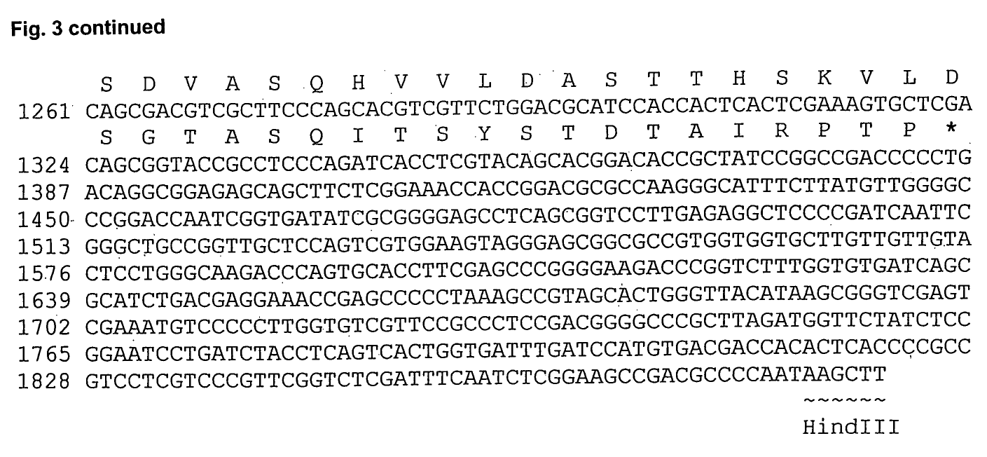

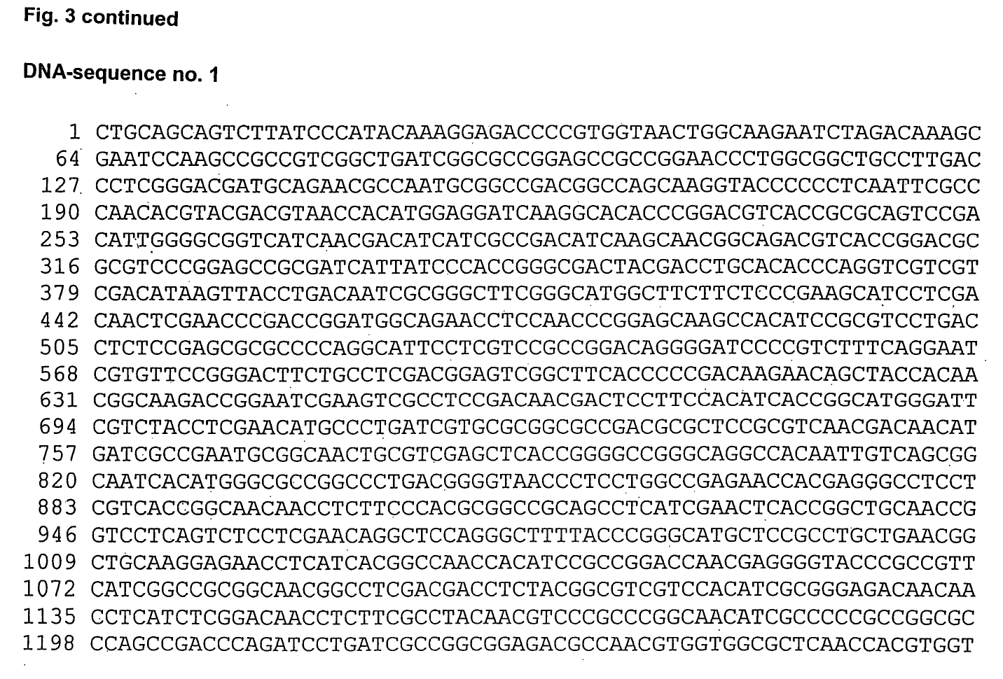

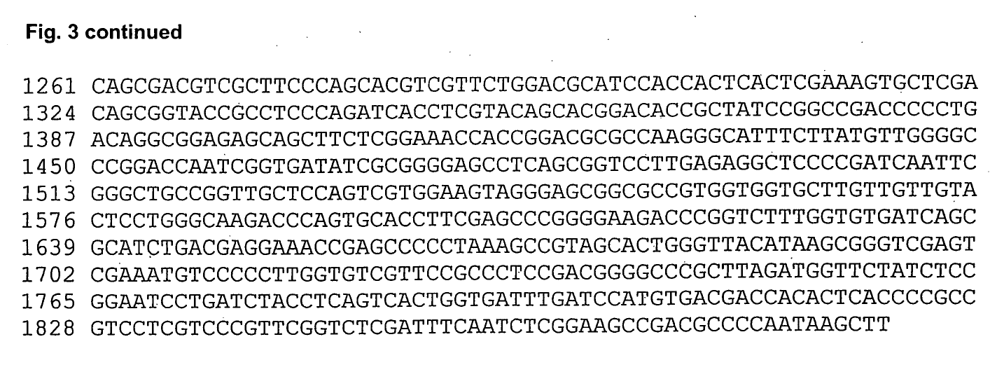

FIG. 3 DNA sequence no. 1 and the amino acid sequence derived therefrom of the expression matrix MSiftPH with the region which codes for active inulase II;

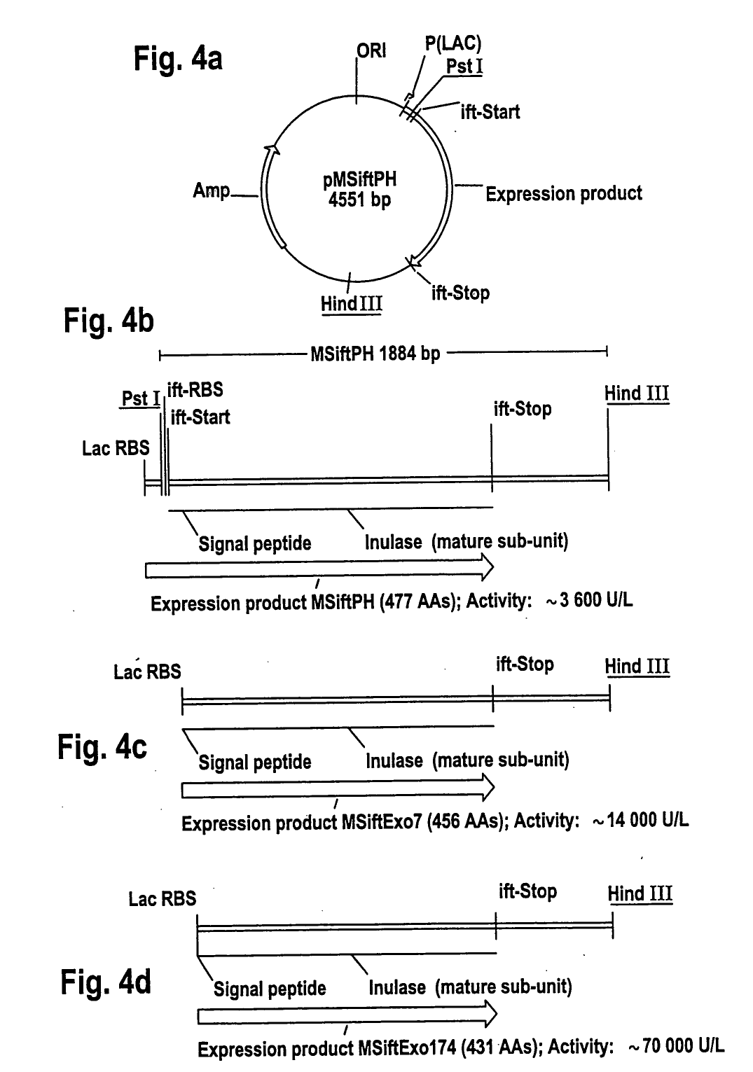

FIG. 4 the gene map of the plasmid pMSiftPH and modified DNA sequences derived therefrom which code for inulase II;

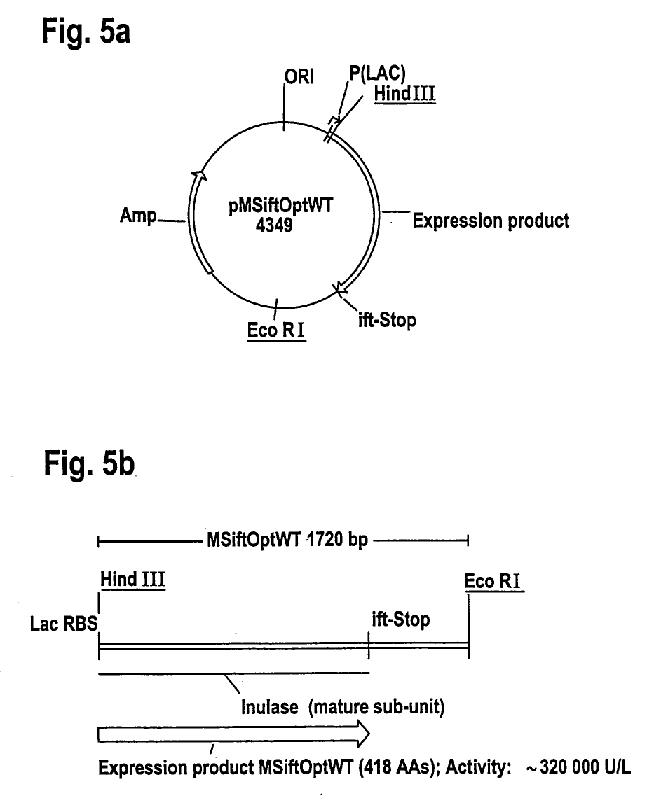

FIG. 5 the gene map of the plasmid pMSiftOptWT;

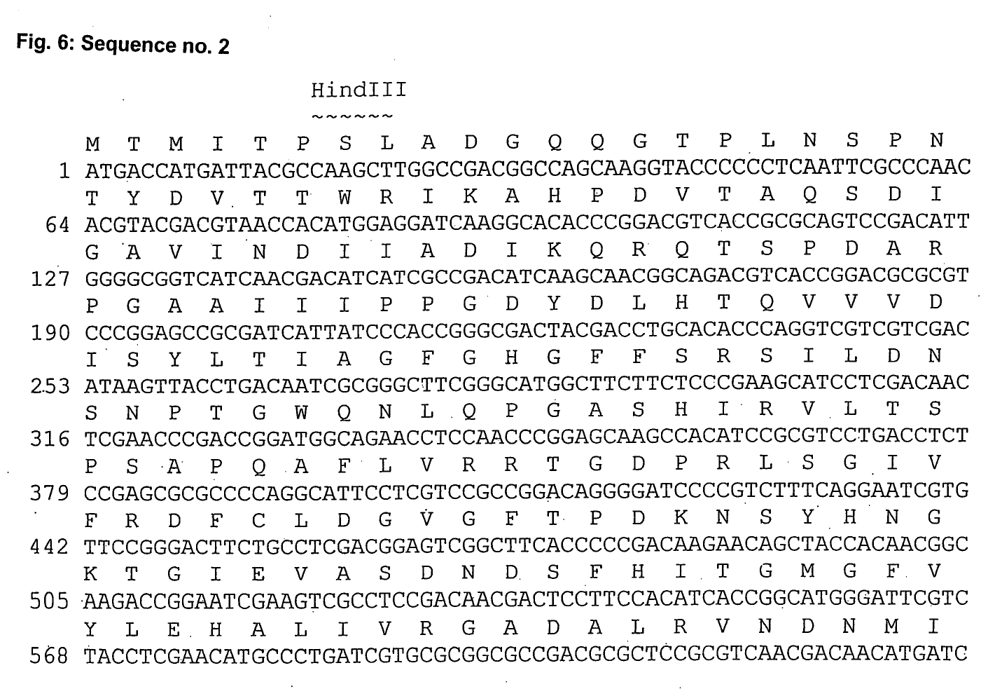

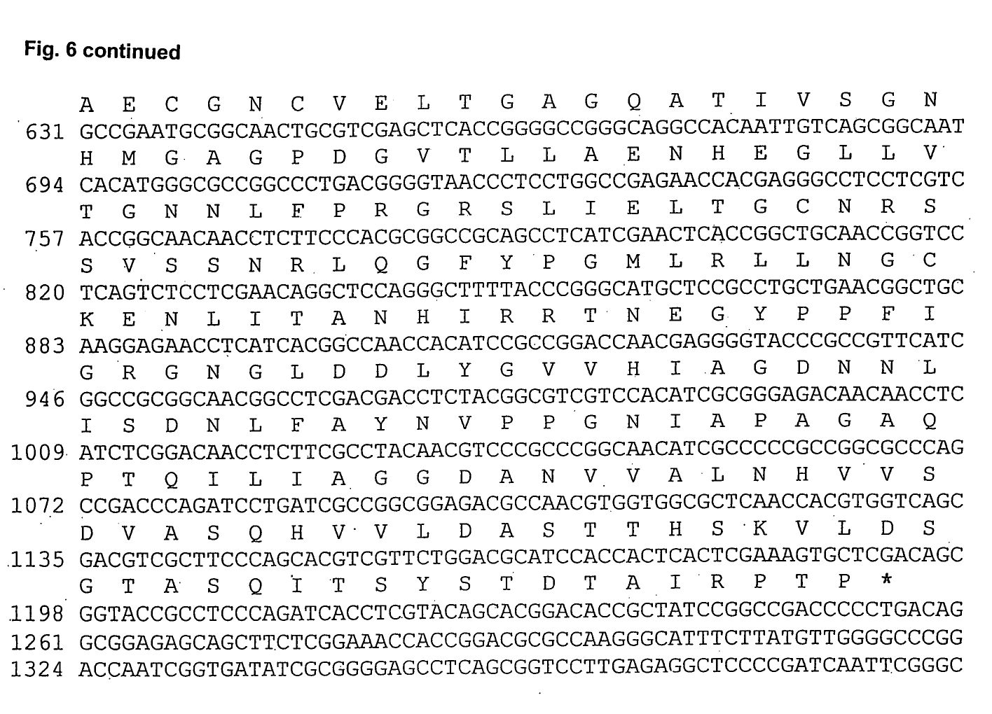

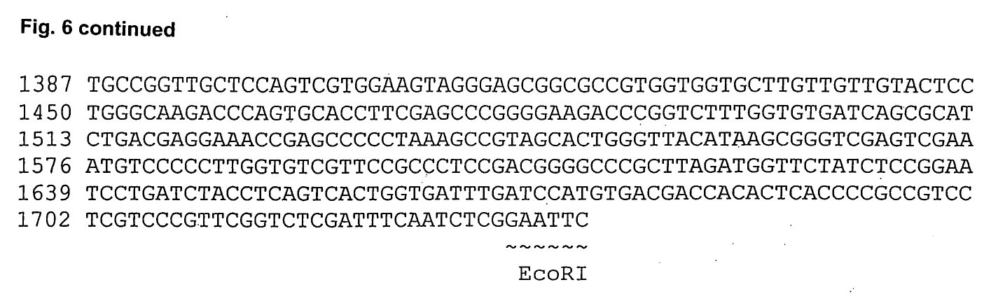

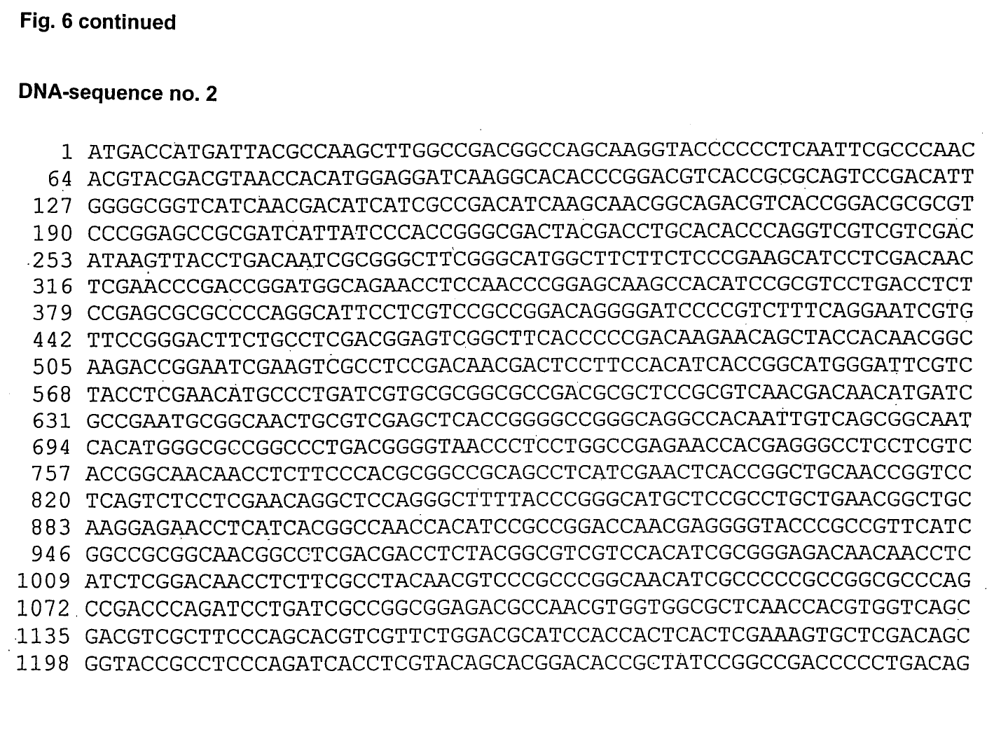

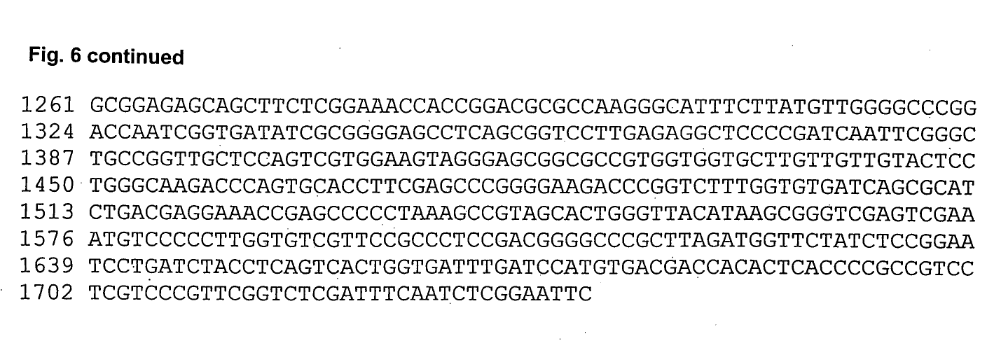

FIG. 6 DNA sequence no. 2 of the expression matrix MSiftOptWT and the amino acid sequence derived therefrom; and

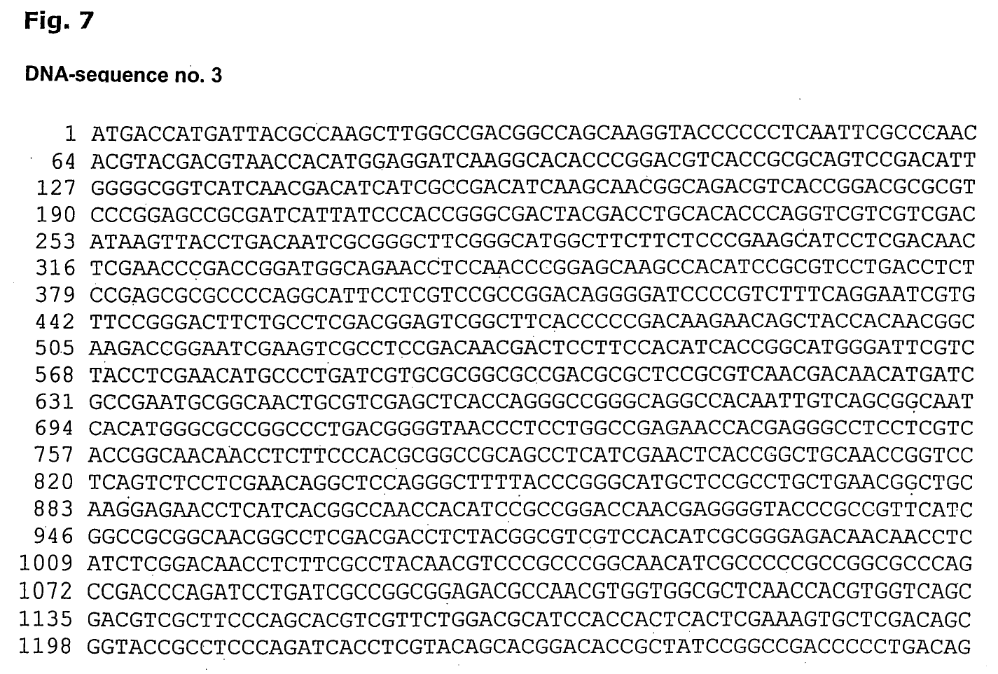

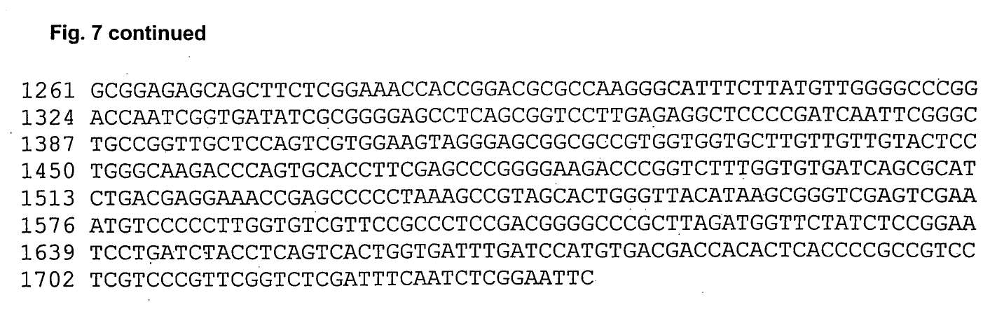

FIG. 7 DNA sequence no. 3 of the plasmid pMSiftOptR.

The continuations of FIGS. 3 and 6 and FIG. 7 furthermore show the coding strand in the 5′-3′ direction (from left to right) in a separate diagram.

The present invention also includes DNA sequences which represent, for example, a fragment, derivative or allelic variant of the DNA sequences described above which code for an enzyme with inulase II activity. The term derivative in this connection means that the sequences differ from the DNA sequences described above at one or more positions but have a high degree of identity to these sequences. A high degree of identity here means a sequence of identity of more than 72.3%, including the region which codes for the signal sequence, and/or more than 74.3% for the region which codes for the mature sub-unit, preferably above 80% and particularly preferably above 90% and in particular at least 95% for the sequence including the signal sequence and/or for the sequence of the mature sub-unit.

The present invention furthermore also includes DNA sequences, the complementary strand of which hybridizes with one of the abovementioned DNA sequences according to the invention and which code for an enzyme with inulase II activity.

In the context of the present invention, the term “hybridization” means a hybridization under conventional hybridization conditions. This is preferably understood as hybridization under stringent conditions.

The invention includes in particular DNA sequences which have the region according to one of sequences no. 1 to 3 which codes for the mature sub-unit, or a modification thereof as described above.

The invention correspondingly also includes enzymes with inulase II activity which can be obtained by expression of a DNA sequence according to the invention, and modifications of such enzymes with an identity of more than 74.9%, including the signal peptide, and/or more than 77.8% for the mature sub-unit.

The DNA sequence shown in sequence no. 1 is a genomic sequence which comprises a coding region for an enzyme with inulase II activity from a microorganism Arthrobacter sp. Bu0141.

With the aid of these sequences, it is now possible for the expert to isolate homologous sequences from other Arthrobacter species or strains. This can be carried out, for example, with the aid of conventional methods, such as screening of gene libraries with suitable hybridization probes.

The DNA sequences according to the invention code for an enzyme with inulase II activity.

The microorganism Arthrobacter sp. Bu0141, the abovementioned plasmids and recombinant E. coli with plasmids pMSiftOptWT and pMSiftOptR have been deposited at the Deutsche Sammlung von Mikroorganismen und Zellkulturen GmbH [German Collection of Microorganisms and Cell Cultures GmbH] under the following numbers and are also subject matter of the invention:

| | | |
|---|---|---|
| Plasmid | pMSiftPH | DSM 13460 |
| Plasmid | pMSiftOptR | DSM 13461 |
| Plasmid | pMSiftOptWT | DSM 13462 |
| E. coli | pMSiftOptWT | DSM 13463 |
| Arthrobacter | sp.Bu0141 | DSM 13464 |
| E. coli | pMSiftOptR | DSM 13645. |

This Arthrobacter strain, called Bu0141 in the following, was isolated from a soil sample and has not been able to be assigned to any of the species described to date. The properties of the strain Bu0141 are described in more detail below.

The microorganism forms coryneform rods, is Gram-positive and strictly aerobic and forms no acid or gas from glucose.

| | |
|---|---|
| Mobility | + |
| Spores | − |
| Catalase | + |
| meso-Diaminopimelic | no |
| acid in the cell wall: | |
| Peptidoglycan type | A3α, L-Lys L-Ala₂₋₃ |

The sequencing of the region with the highest variability (16S rDNA sequence) gave as the highest value 97.8% agreement with Arthrobacter globiformis. It can be concluded from the more than 2% differences in the 16S rDNA sequences that the microorganism is a representative of a species which is indeed closely related to A. globiformis but has not yet been described, and furthermore is not pathogenic.

It has been found that this strain can produce an enzyme with inulase II activity which is stable at elevated temperature over a long period of time. It has thus been found that the enzyme is stable at 60° C. for 140 hours with 100% residual activity.

A DNA sequence (sequence no. 1) which comprises the region which codes for the enzyme with inulase II activity was isolated from this Arthrobacter sp. Bu0141.

For the isolation, the ift gene (codes for inulase) was cloned from Arthrobacter sp. Bu0141 in λ phages, sub-cloned in E. coli and isolated in its complete length on two Bam H1 fragments. The gene map of these fragments, which have been called MSiftBH2 and MSiftBH1, is shown in FIG. 2.

The fragment MSiftBH2 has a length of approximately 3.2 kbp, one part coding for the N-terminal half of the ift gene. The fragment MSiftBH1 has a length of approx. 2.8 kbp, one part coding for the C-terminal half of the ift gene. The two Bam HI fragments were isolated from the complete genomic DNA of Arthrobacter sp. Bu0141.

The singular restriction sites Pst I and Hind III, which serve for construction of an expression matrix as described below, are indicated in the gene map shown in FIG. 2. The putative ribosome binding site ift-RBS and the start and stop codon (ift-start and ift-stop) which demarcate the coding region are also marked in the gene map.

FIG. 3 shows DNA sequence no. 1 and above this the amino acid sequence derived therefrom of the Pst I/Hind III fragment identified in the gene map in FIG. 2 with the ift gene and its surroundings from Arthrobacter sp. Bu0141. This fragment is called expression matrix MSiftPH in the following.

Expression matrix MSiftPH contains 1,884 nucleotides.

The enzyme with inulase II activity is coded by 1,350 nucleotides and comprises 450 amino acids. The first 40 amino acids serve as the signal peptide and ensure transport of the expressed enzyme from the cell. This signal peptide is cut off during or after transport of the enzyme from the cell in Arthrobacter sp. Bu0141. The mature sub-unit of the enzyme with inulase II activity itself comprises 410 amino acids.

The putative ribosome binding site ift-RBS with the start codon GTG, the stop codon (*) and the presumed cleavage site between the signal peptide and the coding region of the mature sub-unit (▾) are furthermore identified in the sequence shown in FIG. 3.

Starting from the fragment MSiftPH, foreign expression systems have now been developed according to the invention, which can be introduced into a host organism and can effect expression of an enzyme with inulase II activity in this host organism.

For this, the above DNA fragment MSiftPH or parts thereof which contain the coding region for the enzyme with inulase II activity were linked to the elements which are suitable for the particular host organism and control transcription, such as promoter and stop codon, it being possible for the DNA sequence to be modified before or after the linking if required.

For example, all or part of the signal sequence was removed from the coding DNA, since as a rule this can be neither recognized by a host organism for export from the cell nor cleaved posttranslationally.

It has been found here that by shortening or complete removal of the signal sequence, a significant increase in the enzyme activity can be effected. The results of these deletion experiments are described in the following.

With the aid of DNA sequence 1 shown under FIG. 3 or parts thereof which contain the coding region for the enzyme with inulase II activity, it is possible to modify microorganisms to the extent that they express active inulase II.

For preparation for introduction of foreign genes into microorganisms, a large number of cloning vectors which contain the elements for control of expression required for a particular microorganism are available. The desired sequence can be introduced into the vector at an appropriate restriction cleavage site. Any plasmid DNA sequence can be cloned into the same vector or into other plasmids by this procedure. The techniques, vectors and appropriate control elements are known per se and can easily be chosen and/or adapted for the particular host organism to be transformed.

The production of recombinant host organisms according to the invention which contain a DNA sequence according to the invention and have the ability to express active inulase II is described in the following by the example of transformation of E. coli, expression constructs which contain DNA sequence I shown in FIG. 3 or parts thereof with the region which codes for inulase II being introduced into the microorganism. pUC 18 and pUC 19 were used as vectors for the following example. The DNA fragments MSiftPH according to FIG. 3 and modifications thereof which contained the region which codes for active inulase II were introduced into these vectors and the corresponding enzyme activity of E. coli transformed therewith was investigated.

FIG. 4 shows the gene map of the inulase expression construct pMSiftPH obtained, the expression matrix MSiftPH, which is shown in FIG. 3, having been integrated into the commercially obtainable vector pUC 18. The expression construct pMSiftPH was transformed into E. coli. The quality of the expression construct was checked in the inulase activity test described in the following. Transformants with the expression construct pMSiftPH showed a significant inulase activity of about 3,600 U/l.

Deletion experiments were undertaken in order to investigate the influence of the signal peptide on the enzyme activity. It was shown in these that by shortening the signal peptide at the DNA level it was possible to increase the inulase activity of the expression construct pMSiftPH 20-fold. Thus, an expression product (enzyme) with only 456 amino acids compared with 477 amino acids for the expression product (enzyme) of MSiftPH shows an activity of about 14,000 U/l (FIG. 4c) and the corresponding expression product (enzyme) with 431 amino acids shows an activity of about 70,000 U/l (FIG. 4d).

A DNA sequence (sequence no. 2) which codes for active inulase II and in which the DNA sequence which codes for the signal peptide has been completely removed is shown in FIG. 6. Nucleotide sequence no. 2 shown in FIG. 6 is called expression matrix MSiftOptWT in the following. In this sequence the region which codes for the mature inulase sub-unit without the signal peptide starts at nucleotide position 25.

The expression matrix MSiftOptWT was tested for efficiency in several vectors. It was found here that it was possible to achieve an increase in inulase activity by a factor of 2 to 3 solely by cloning the same expression matrix from plasmid pUC 18 to pUC 19.

The expression construct pMSiftOptWT was prepared from the expression matrix MSiftOptWT and plasmid pUC 19 by integrating the MSiftOptWT fragment, optimized to a nucleotide, directly into the reading frame in pUC 19 which starts at the Lac RBS via the synthetically produced Hind III or Eco RI cleavage sites.

The gene map of the pMSiftOptWT expression constructs obtained, of the combination of expression matrix MSiftOptWT and the plasmid pUC 19, is shown in FIG. 5. The inulase activity of an E. coli transformed with the vector pMSiftOptWT was greater than 320,000 U/l.

A fusion protein with the amino acid sequence shown in FIG. 6, which comprises 418 amino acids, was obtained as the expression product, the enzyme with inulase activity starting at amino acid position 9 ADGQQ . . .

FIG. 7 shows DNA sequence no. 3 of a plasmid pMSiftOptR which differs from DNA sequence no. 2 of plasmid pMSiftOptWT in one nucleotide at position 661 in that nucleotide G of sequence no. 2 has been replaced by nucleotide A in sequence no. 3, as a result of which R (Arg) is incorporated instead of G (Gly) at position 221 in the corresponding amino acid sequences. This minor modification causes an increase in activity to 435,000 U/l, that is to say an increase by a factor of 1.35.

The procedure for the experiments and the results of an inulase activity test carried out with the expression products (enzymes) obtained are described in the following.

Procedure for the inulase activity test

The test described in the following served merely for a rapid comparative analysis, and it is to be expected that the values for the enzyme activity will be several times higher on a preparative scale under optimized conditions, such as larger number of cells, more effective cell disruption etc.

The strains were cultured by inoculating 5 ml Luria-Bertani medium, to which 60 μg/ml ampicillin had been added, with a single colony of E. coli, which had been transformed with the particular expression construct (plasmid), and shaking the culture for 16 hours at 37° C. and 170 rpm.

The host organism used was an E. coli strain from Stratagene® {E. coli XL1-blue MRF′ Kan: Δ(mcrA)183 Δ(mcrCB-hsdSMR-mrr)173 endA1 supE44 thi-1 recA1 gyrA96 relA1 lac [F′ proAB lacI^{q}ZΔM15 Tn5 (Kan′)]^{c}}.

The inulase II expression was intracellular; the enzyme reaction and DFA III formation took place in the cell-free extract after disruption of the cells.

For the activity test, in each case 0.5 ml of the fresh expression culture described above was used, 0.5 ml of the culture being pelleted, the supernatant being discarded and the cells being resuspended in 5 ml of cooled 0.9% NaCl solution.

The cell disruption was carried out by means of ultrasonification (KE 76, cont. 50%, 60 sec; Bandelin, Sonopulus HD 200).

1 ml of the disrupted cells was removed and pelleted in a bench centrifuge for 10 min (20,000×g). 100 μl of the enzyme-containing supernatant were transferred to 1,000 μl of a 10% inulin solution (pH 5.5) and incubated for 30 min at 50° C.

The enzyme reaction was stopped by heating to 100° C. for 10 min and the solution was centrifuged in a bench centrifuge (10 min, 20,000×g).

100 μl of the supernatant, which contained the product DFA III, were transferred into 1,000 μl HPLC eluent and the product DFA III was measured by means of HPLC.

A value of product formation of approximately 2.6 g/l resulted here for the clone of the expression construct pMSiftOptWT, which corresponded to an enzyme activity of approx. 323,000 U/l (one unit=1 μmol/min).

For the clone of the expression construct pMSiftOptR, which differs from expression construct pMSiftOptWT in a single nucleotide at position 661 by replacement of G (sequence no. 2) by A (sequence no. 3), a value of 3.5 g/l DFA III was found for the product formation, which corresponded to an enzyme activity of approx. 435,000 U/l. Compared with the expression construct pMSiftOptWT, an increase of 1.35-fold is thus observed.

The corresponding enzyme activities for clones of the expression construct with plasmid pUC18 and of expression matrix MSiftPH with a complete signal sequence corresponding to an expression product with 477 amino acids, with a shortened signal sequence corresponding to an expression product with 456 amino acids or corresponding to an expression product with 431 amino acids, and with expression matrix MSiftOptWT without a signal sequence were approx. 3,500, approx. 14,000, approx. 70,000 and approx. 120,000 U/l.

On the basis of their high heat stability, the enzymes with inulase II activity obtainable from Arthrobacter sp. Bu0141 and the DNA sequences isolated or derived therefrom which code for an enzyme with inulase II activity are outstandingly suitable for a process for the enzymatic decomposition of inulin for the production of difructose anhydride III. Due to the possibility described of transforming and expressing these DNA sequences in generally available host organisms, tailor-made recombinant microorganisms with a high enzyme activity can be obtained, the enzymes expressed having a high heat stability and therefore being able to decompose inulin to difructose anhydride III efficiently.

1. DNA sequence coding for an enzyme with inulase II activity chosen from a DNA sequence with a nucleotide sequence according to one of sequences no. 1, no. 2 or no. 3, a DNA sequence which comprises the region of sequences no. 1, no. 2 or no. 3 which codes for an enzyme with inulase II activity;
a DNA sequence which codes an enzyme with inulase II activity which comprises the amino acid sequence given for sequences no. 1, no. 2 or no. 3; and
sequences homologous to DNA sequences no. 1, 2 or 3 which have an identity of more than 72.3%, including the region which codes for the signal sequence, and/or more than 74.3% for the region which codes for the mature sub-unit.

2. Vector comprising a DNA sequence according to claim 1.

3. Vector according to claim 2,
characterized in that
the vector is a plasmid pUC 18 or pUC 19.

4. Plasmid chosen from plasmids with deposit number DSM 13460, DSM 13461 and DSM 13462.

5. Microorganism
characterized in that
the microorganism contains a DNA sequence according to claim 1, a vector according to one of claims 2 or 3 or a plasmid according to claim 4.

6. Microorganism according to claim 5,
characterized in that
the microorganism is an E. coli with deposit number DSM 13463 or DSM 13465.

7. Microorganism of the species Arthrobacter sp. with deposit number DSM 13464.

8. Enzyme with inulase II activity, obtainable by expression of one of the DNA sequences according to claim 1.

9. Process for the enzymatic decomposition of inulin to difructose anhydride III,
characterized in that
there is used an enzyme with inulase II activity which is obtainable via one of the DNA sequences according to claim 1.

10. Process according to claim 9,
characterized in that
the DNA sequence is introduced into a microorganism and expressed there.

11. Process according to one of claims 9 or 10,
characterized in that
a plasmid chosen from plasmids with deposit number DSM 13460, DSM 13461 and DSM 13462 or a microorganism chosen from microorganisms with deposit number DSM 13463, DSM 13464 and DSM 13465 is used for the process.
